(19) United States
(12) Patent Application Publication
(10) Pub. No.: US 2026/0132250 A1
(43) Pub. Date: May 14, 2026

(54) METHOD FOR PRODUCING POLYHYDROXYALKANOATE AND USE THEREOF

(71) Applicant: KANEKA CORPORATION, Osaka-shi (JP)

(72) Inventor: Masaru HIRANO, Takasago-shi (JP)

(73) Assignee: KANEKA CORPORATION, Osaka-shi (JP)

(21) Appl. No.: 18/993,608

(22) PCT Filed: Jun. 21, 2023
(86) PCT No.: PCT/JP2023/022865

§ 371 Date: Jan. 13, 2025

(30) Foreign Application Priority Data
Aug. 5, 2022 (JP) 2022-125872

(51) Int. Cl. C08G 63/89

(52) U.S. Cl. CPC C08G 63/89

(57) ABSTRACT

The object of the present invention is to provide a method for producing a PHA having a low water content. The object is attained by providing a method for producing a PHA, including a filtration step of filtering an aqueous PHA suspension having a pH of 2.5 to 5.5, by filter-pressing with use of a filter medium having an air permeability of 0.1 cm³/cm²/min to 2.5 cm³/cm²/min, the aqueous PHA suspension having a liquid density of 0.50 g/mL to 1.08 g/mL in the filtration step, the filtration step including a compression step and an air blow step.

### TECHNICAL FIELD

The present invention relates to a method for producing a polyhydroxyalkanoate and use of the polyhydroxyalkanoate.

### BACKGROUND ART

A polyhydroxyalkanoate (hereinafter, also referred to as a “PHA”) is known to be biodegradable.

A PHA produced by microorganisms is accumulated in the microbial cells of the microorganisms. Accordingly, in order to use a PHA as plastic, it is necessary to carry out a step of isolating a PHA from the microbial cells of the microorganisms and purifying the PHA. In the step of isolating and purifying a PHA, the microbial cells of PHA-containing microorganisms are disrupted or the microorganism-derived components, excluding the PHA, are solubilized, before from an aqueous suspension thus obtained, the PHA is extracted. In this extraction, isolation operations such as, for example, centrifugation, filtration, and drying are carried out.

As the PHA production method in which filtration is used, disclosed in, for example, Patent Literature 1 is a PHA production method including: a step of inoculating a fermentation medium with a species of PHA fermenter to cause fermentation; a step of subjecting a fermentation liquid to solid-liquid separation to obtain a fermentation supernatant liquid and a microbial cell precipitate; and a step of precipitating microbial cells and disrupting the cell walls of the microbial cells, and subjecting the disrupted cell walls to plate-and-frame filtration with use of a precoated filter, to obtain the PHA.

### CITATION LIST

### Patent Literature

[Patent Literature 1] Specification of Chinese patent No. 111500650

### SUMMARY OF INVENTION

### Technical Problem

However, the PHA obtained by the above-described technique has a high water content. Accordingly, in a subsequent stage, energy for volatilizing water is required. Therefore, the PHA has room for further improvement.

An object of the present invention is to provide a method for producing a PHA having a low water content and a PHA agglutinate having a low water content.

### Solution to Problem

The inventor of the present invention conducted diligent studies in order to attain the above object, and consequently obtained a novel finding that it is possible to obtain a PHA having a low water content, by, in production of the PHA, carrying out a specific filtration step in which the pH of an aqueous PHA suspension, the air permeability of a filter medium, and the liquid density of the aqueous PHA suspension are controlled. As a result, the inventor of the present invention completed the present invention.

Therefore, an aspect of the present invention is a method for producing a PHA, including: a filtration step of filtering an aqueous PHA suspension having a pH of 2.5 to 5.5, by filter-pressing with use of a filter medium having an air permeability of 0.1 cm³/cm²/min to 2.5 cm³/cm²/min, the aqueous polyhydroxyalkanoate suspension having a liquid density of 0.50 g/mL to 1.08 g/mL in the filtration step, the filtration step including a compression step and an air blow step (hereinafter, the method is referred to as “the present production method”).

An aspect of the present invention is a PHA agglutinate having a water content of 5.0% to 25.0% (W. B.) (hereinafter, the PHA agglutinate is referred to as “the present PHA agglutinate”).

### Advantageous Effects of Invention

An aspect of the present invention makes it possible to provide (i) a method for producing a PHA having a low water content and (ii) a PHA agglutinate having a low water content.

### BRIEF DESCRIPTION OF DRAWINGS

FIG. 1 is a photograph of liquids having passed through filter cloths in Example 1-1 and Comparative Example 2 in accordance with an embodiment of the present invention.

### DESCRIPTION OF EMBODIMENTS

The following description will discuss an embodiment of the present invention in detail. Note that a numerical range expressed as “A to B” means “not less than A and not more than B”, unless otherwise specified herein.

### [1. Outline of the Present Invention]

There is a problem that a PHA produced within a microbial cell is difficult to filter because the particle diameter of the PHA is approximately 1 μm to 2 μm. Further, the PHA is recovered mainly by centrifugation, and the form of separation is an aqueous suspension. Thus, the PHA is recovered in a state where a large amount of water is contained. In order to separate the PHA from the water and recover the PHA, it is necessary to evaporate the water contained in the aqueous suspension. This presents a problem such as the need for a large amount of energy. As a PHA production method in which filtration is used, for example, the above-described method disclosed in Patent Literature 1 is known. In the technique disclosed in Patent Literature 1, a filter medium is precoated, and then filtration is carried out before purification (in a state where many biological residues remain). Therefore, it was found that the technique has a problem that an operation is complicated and an obtained PHA agglutinate contains many impurities. Moreover, it was found that the technique disclosed in Patent Literature 1 has a problem that a water content is not sufficiently lowered.

Under the circumstances, the inventor of the present invention conducted diligent studies from the viewpoint of reducing the water content of a PHA, and consequently found for the first time that it is possible to obtain a PHA having a low water content by, in production of the PHA, carrying out a specific filtration step in which the pH of an aqueous PHA suspension, the air permeability of a filter medium, and the liquid density of the aqueous PHA suspension are controlled. Specifically, the inventor of the present invention developed a technique of (i) adjusting the pH of an aqueous PHA suspension so that the pH falls within the range in which a PHA agglutinates and (ii) then recovering, by filtration, the PHA that has agglutinated. By the present production method, it is possible to provide a PHA having a low water content. Furthermore, by the present production method, it is possible to, without adding an impurity such as a filter aid, produce a PHA having a low water content, and thus possible to reduce impurities. Therefore, the present production method is extremely advantageous in industrial production of a PHA.

In addition, by a configuration as described above, it is possible to reduce the quantity of heat, time, and costs, i.e., energy, which are required in a drying step carried out after filtration. Thus, the present invention is capable of contributing to achieving the sustainable development goals (SDGs), for example, Goal 7 “Ensure access to affordable, reliable, sustainable and modern energy for all” etc. The present invention will be described below in detail.

### [2. Method for Producing PHA]

The present production method includes the following step:
a filtration step of filtering an aqueous PHA suspension having a pH of 2.5 to 5.5, by filter-pressing with use of a filter medium having an air permeability of 0.1 cm³/cm²/min to 2.5 cm³/cm²/min, the aqueous polyhydroxyalkanoate suspension having a liquid density of 0.50 g/mL to 1.08 g/mL in the filtration step, the filtration step including a compression step and an air blow step (hereinafter, the filtration step is referred to as a “step (d)”).

In an embodiment of the present invention, the present production method preferably includes, in addition to the above step (d), at least one of the following steps.
Step (a): a step of disrupting and solubilizing a cell-derived component of a microbial cell which contains a PHA, the cell-derived component being a component other than the PHA, the PHA having a volume median diameter of 0.5 μm to 5.0 μm (the step (a) is also referred to as a “solubilization step”).
Step (b): a step of recovering the aqueous PHA suspension by centrifugation, after the step (a) (the step (b) is also referred to as a “recovery step”).
Step (c′): a step of adjusting the pH of the aqueous PHA suspension obtained in the step (b) to 2.5 to 5.5 (the step (c′) is also referred to as an “adjustment step”).
Step (c): a step of heat-treating the aqueous PHA suspension so that the aqueous PHA suspension has a temperature of 60° C. to 120° C. (the step (c) is also referred to as a “heat treatment step”).
Step (e): a step of drying the PHA obtained in the step (d) at 20° C. to 100° C. (the step (e) is also referred to as a “drying step”).
Step (f): a step of redispersing the dried PHA in an aqueous solvent (the step (f) is also referred to as a “redispersion step”).

In the present production method, the above steps are preferably carried out in the order of the steps (a), (b), (c′), (c), (d), (e), and (f). However, the order can be changed as appropriate, according to purposes. For example, the order of the steps (a) and (b) can be exchanged so that the steps (b) and (a) are carried out in this order, and the order of the steps (c′) and (c) can be exchanged so that the steps (c) and (c′) are carried out in this order. Furthermore, according to purposes, each of the steps (a), (b), (c′), and (c) can be carried out twice or more. That is, for example, the steps (b), (a), and (b) can be carried out in this order or the steps (c′), (c), and (c′) can be carried out in this order. Note that, in the present specification, an aqueous suspension containing at least a PHA may be abbreviated to an “aqueous PHA suspension”.

### (Step (d))

First, the step (d), which is a characteristic configuration of the present invention, is described.

In the step (d) of the present production method, an aqueous PHA suspension having a pH of 2.5 to 5.5 is filtered by filter-pressing with use of a filter medium having an air permeability of 0.1 cm³/cm²/min to 2.5 cm³/cm²/min. In the step (d), the aqueous PHA suspension has a liquid density of 0.50 g/mL to 1.08 g/mL in the filtration step. In the step (d), such a filter-pressing filtration step includes a compression step and an air blow step. By the step (d), a PHA having a low water content is obtained.

The filter-pressing filtration step of the step (d) includes a compression step and an air blow step. In the compression step, a PHA cake is squeezed by compression so that water is squeezed out from the PHA cake. In the air blow step, water is pressed out from the PHA cake by air-blown air. Through such a two-step dehydration step, that is, the compression step and the air blow step, it is possible to obtain a PHA agglutinate having a low water content.

In an embodiment of the present invention, the air blow step can be a through air blow step.

In an embodiment of the present invention, the filter-pressing filtration step can include a step of supplying a stock solution (aqueous PHA suspension) to a filter chamber, before the compression step.

A pressure in the compression step is preferably 0.2 MPa to 1.0 MPa, more preferably 0.25 MPa to 0.9 MPa, and even more preferably 0.3 MPa to 0.8 MPa. In a case where the pressure in the compression step is 0.2 MPa to 1.0 MPa, there is an advantage that the PHA agglutinate has a decreased water content.

An air blow pressure in the air blow step is not particularly limited, and, for example, 0.01 MPa to 1.5 MPa, preferably 0.05 MPa to 1.3 MPa, and more preferably 0.10 MPa to 1.0 MPa. In a case where the air blow pressure falls within the above range, there is an advantage that the PHA agglutinate has a decreased water content.

An air blow duration can be set, as appropriate, in accordance with the air blow pressure, and, for example, 1 minute to 50 minutes and preferably 5 minutes to 40 minutes.

A device which carries out filter-pressing filtration is not particularly limited, and can be any publicly known device.

The filter medium used in the step (d) is not particularly limited, but can be selected from among various materials such as, for example, paper, filter cloths (woven cloths, non-woven cloths), screens, sintered plates, porcelain filters, polymer membranes, punching metals, and wedge wires. A filter cloth is preferably used from the viewpoint of price and case of washing.

In the present specification, the amount (cm³) of air that passes through a unit area (cm²) of the filter medium per minute is referred to as an air permeability. In the step (d), the air permeability is 0.1 cm³/cm²/min to 2.5 cm³/cm²/min, preferably 0.2 cm³/cm²/min to 2.0 cm³/cm²/min, more preferably 0.3 cm³/cm²/min to 1.8 cm³/cm²/min, even more preferably 0.5 cm³/cm²/min to 1.5 cm³/cm²/min, and particularly preferably 0.8 cm³/cm²/min to 1.2 cm³/cm²/min. In a case where the air permeability falls within the above range, there is an advantage that a rate of leakage of the PHA into a filtrate is low. Note that the air permeability in the filtration step of the present production method is measured by the method described in Examples.

In the step (d), the aqueous PHA suspension has a liquid density of 0.50 g/mL to 1.08 g/mL, preferably 0.60 g/mL to 1.05 g/mL, more preferably 0.70 g/mL to 1.03 g/mL, and even more preferably 0.80 g/mL to 1.02 g/mL. In a case where the liquid density of the aqueous PHA suspension falls within the above range, there is an advantage that a filtrate permeation rate is high and the water content of the PHA agglutinate is low. An inferred reason why the filtrate permeation rate decreases in a case where the liquid density is low is as follows: the aqueous PHA suspension has an increased viscosity by containing air, and the air and the PHA interact with each other and consequently the viscosity increases. The liquid density of the aqueous PHA suspension can be adjusted by, for example, including air into the aqueous PHA suspension. In a case where the amount of the air is increased, the liquid density of the aqueous PHA suspension decreases. In a case where the amount of the air is reduced, the liquid density of the aqueous PHA suspension increases.

The filtrate in the step (d) has a solid content concentration of preferably not more than 1,000 mg/L, more preferably not more than 500 mg/L, even more preferably not more than 200 mg/L, and particularly preferably not more than 100 mg/L. In a case where the solid content concentration of the filtrate falls within the above range, there is an advantage that the rate of recovery of the PHA agglutinate is high. Note that the lower limit is not particularly limited, and may be, for example, 0 mg/mL. Note that the solid content concentration of the filtrate in the filtration step of the present production method is measured by the method described in Examples.

In an embodiment of the present invention, the filter medium is preferably not precoated. In a case where the filter medium is not precoated, there is an advantage that an operation is uncomplicated and it is possible to reduce the amount of impurities in the PHA.

The aqueous PHA suspension in the step (d) has a temperature (filtration temperature) of preferably 20° C. to 95° C., more preferably 25° C. to 90° C., even more preferably 30° C. to 85° C., and particularly preferably 35° C. to 70° C. In a case where the temperature of the aqueous PHA suspension falls within the above range, there is an advantage that the filtrate permeation rate is high. An inferred reason for an increase in the filtrate permeation rate is that an increase in the temperature causes an increase in the viscosity and, at the same time, causes an increase in the particle diameter.

In a case where the present production method includes the heat treatment step of the step (c), the temperature of the aqueous PHA suspension during the filtration is lower, by preferably not less than 5° C., more preferably not less than 8° C., even more preferably not less than 10° C., and particularly preferably not less than 12° C., than the temperature of the aqueous PHA suspension after the heat treatment step. In a case where the temperature of the aqueous PHA suspension during the filtration falls within the above range, there is an advantage that it is possible to filer the PHA at a high filtrate permeation rate. A method for decreasing the temperature after the heat treatment step is not particularly limited, and examples thereof include: cooling by a cooling device; and leaving the aqueous PHA suspension to cool.

Note that, to the “pH” in the step (d), the description given below in connection with the step (c′) applies.

### (Step (a))

In the step (a) of the present production method, a cell-derived component, other than the PHA, of a microbial cell which contains the PHA is disrupted and solubilized. By disrupting and removing such a cell-derived impurity (cell wall, protein, or the like) through the step (a), it is possible to efficiently recover, from the microbial cell, the PHA having a volume median diameter of 0.5 μm to 5.0 μm.

### <PHA>

As used herein, the term “PHA” is a generic term for polymers each of which contains a hydroxyalkanoic acid as a monomer unit. A hydroxyalkanoic acid which constitutes the PHA is not particularly limited, but examples thereof include 3-hydroxybutanoic acid, 4-hydroxybutanoic acid, 3-hydroxypropionic acid, 3-hydroxypentanoic acid, 3-hydroxyhexanoic acid, 3-hydroxyheptanoic acid, and 3-hydroxyoctanoic acid. The polymers may be homopolymers or copolymers each of which contains two or more types of monomer units.

More specifically, examples of the PHA include poly(3-hydroxybutyrate) (P3HB), poly(3-hydroxybutyrate-co-3-hydroxyhexanoate) (P3HB3HH), poly(3-hydroxybutyrate-co-3-hydroxyvalerate) (P3HB3HV), poly(3-hydroxybutyrate-co-4-hydroxybutyrate) (P3HB4HB), poly(3-hydroxybutyrate-co-3-hydroxyoctanoate) (P3HB3HO), poly(3-hydroxybutyrate-co-3-hydroxyoctadecanoate) (P3HB3HOD), poly(3-hydroxybutyrate-co-3-hydroxydecanoate) (P3HB3HD), and poly(3-hydroxybutyrate-co-3-hydroxyvalerate-co-3-hydroxyhexanoate) (P3HB3HV3HH). Among these examples, P3HB, P3HB3HH, P3HB3HV, and P3HB4HB are preferable because they are easy to industrially produce.

Further, P3HB3HH, which is a copolymer of 3-hydroxybutyric acid and 3-hydroxyhexanoic acid, is more preferable from the following viewpoints: (i) by changing the composition ratio of repeating units, it is possible to cause a change in melting point and crystallinity and consequently cause a change in physical property, such as a Young's modulus or heat resistance, of P3HB3HH, and possible to cause P3HB3HH to have a physical property intermediate between those of polypropylene and polyethylene; and (ii) P3HB3HH is a plastic that is easy to industrially produce as described above and has useful physical properties.

According to an embodiment of the present invention, the composition ratio of repeating units of P3HB3HH is such that the composition ratio of a 3-hydroxybutyrate unit to a 3-hydroxyhexanoate unit is preferably 80/20 (mol/mol) to 99.9/0.1 (mol/mol) and more preferably 85/15 (mol/mol) to 97/3 (mol/mol), from the viewpoint of the balance between plasticity and strength. In a case where the composition ratio of the 3-hydroxybutyrate unit to the 3-hydroxyhexanoate unit is not more than 99.9/0.01 (mol/mol), sufficient plasticity is obtained, In a case where the composition ratio is not less than 80/20 (mol/mol), sufficient hardness is obtained.

The volume median diameter of the PHA in the step (a) is preferably not more than 50 times, more preferably not more than 20 times, and even more preferably not more than 10 times the volume median diameter (hereinafter referred to as a “primary particle diameter”) of primary particles of the PHA. In a case where the volume median diameter of the PHA is not more than 50 times the primary particle diameter, the aqueous PHA suspension exhibits more excellent flowability. Thus, the productivity of the PHA tends to further improve.

According to an embodiment of the present invention, the volume median diameter of the PHA is, for example, preferably 0.5 μm to 5.0 μm, more preferably 1.0 μm to 4.5 μm, and even more preferably 1.0 μm to 4.0 μm, from the viewpoint of achieving excellent flowability. The volume median diameter of the PHA is measured with use of a laser diffraction/scatter particle size distribution meter LA-950 manufactured by HORIBA.

Note that, for convenience, the volume median diameter of the PHA in the steps subsequent to the step (a) generally has a value similar to that of the volume median diameter of the PHA which is defined in the step (a). Therefore, the volume median diameter may be measured in any of the steps (a) to (d).

### <Microbial Cell (Microorganism)>

A microorganism used in the step (a) is not particularly limited, provided that the microorganism can produce the PHA within a cell thereof. For example, it is possible to use a microorganism isolated from nature, a microorganism deposited in a depositary institution (for example, IFO and ATCC) for microbial strains, or a mutant, a transformant, or the like that can be prepared from any of these microorganisms. Examples of a microbial cell that produces P3HB, which is an example of the PHA, include Bacillus megaterium, which is the first P3HB-producing microbial cell discovered in 1925, and also include other natural microorganisms such as Cupriavidus necator (former classification: Alcaligenes eutrophus, Ralstonia eutropha) and Alcaligenes latus. These microorganisms are known to have PHAs accumulated within cells thereof.

Examples of a microbial cell that produces a copolymer of hydroxybutyrate and another hydroxyalkanoate, the copolymer being an example of the PHA, include: Aeromonas caviae, which is a P3HB3HV and P3HB3HH-producing microorganism; and Alcaligenes eutrophus, which is a P3HB4HB-producing microorganism. In particular, regarding P3HB3HH, the microbial cell is more preferably Alcaligenes eutrophus AC32 strain (FERM BP-6038), which has introduced therein genes of a group of PHA synthetases, (T. Fukui, Y. Doi, J. Bacteriol., 179, pp. 4821 to 4830 (1997)), or the like, for the purpose of enhancing the productivity of P3HB3HH. Besides the above, the microbial cell may be of a genetically engineered microorganism which has introduced therein various types of PHA synthesis-related genes selected according to a desired PHA to be produced.

### <Disruption and Solubilization of Cell-Derived Component>

In the step (a), a method for disrupting and solubilizing the cell-derived component, other than the PHA, of the microbial cell which contains the PHA is not particularly limited.

According to an embodiment of the present invention, the above disruption and solubilization are carried out with use of, for example, a lytic enzyme and/or a proteolytic enzyme (e.g., alkaline proteolytic enzyme).

As used herein, the term “lytic enzyme” means an enzyme that has the activity of degrading (lysing) the cell wall (for example, peptidoglycan) of the microbial cell.

According to an embodiment of the present invention, the lytic enzyme is not particularly limited, but examples thereof include lysozyme, labiase, β-N-acetylglucosaminidase, an endolysin, and an autolysin. The lytic enzyme is preferably lysozyme from the viewpoint of economic advantage. One of these lytic enzymes may be used alone, or two or more of these lytic enzymes may be used in combination.

As the lytic enzyme, a commercially available lytic enzyme can also be used. Examples of the commercially available lytic enzyme include “Lysozyme” and “Achromopeptidase” manufactured by FUJIFILM Wako Pure Chemical Corporation.

According to an embodiment of the present invention, the optimum pH of the lytic enzyme is not particularly limited, provided that the lytic enzyme has cell wall degradation activity, but is, for example, 5.0 to 11.0, preferably 6.0 to 9.0, and more preferably 6.0 to 8.0.

According to an embodiment of the present invention, the optimum temperature of the lytic enzyme is not particularly limited, but is preferably not higher than 60° C. and more preferably not higher than 50° C., from the viewpoint of not requiring undue heating and accordingly preventing a thermal change (pyrolysis) of the PHA. The lower limit of the optimum temperature is not particularly limited, but is preferably not lower than room temperature (e.g., 25° C.), from the viewpoint of not requiring an undue cooling operation and therefore being economical.

As used herein, the term “alkaline proteolytic enzyme” means a proteolytic enzyme having the activity of degrading protein in an alkaline environment (e.g., in a solution having a pH of 8.5).

According to an embodiment of the present invention, the alkaline proteolytic enzyme is not particularly limited, provided that the alkaline proteolytic enzyme has the activity of degrading protein in an alkaline environment, but examples thereof include serine-specific proteolytic enzymes (e.g., subtilisin, chymotrypsin, and trypsin), cysteine-specific proteolytic enzymes (e.g., papain, bromelain, and cathepsin), and aspartic acid-specific proteolytic enzymes (e.g., pepsin, cathepsin D, and HIV protease). The alkaline proteolytic enzyme is preferably a serine-specific proteolytic enzyme and, in particular, subtilisin (e.g., alcalase), from the viewpoint of economic advantage. One of these alkaline proteolytic enzymes may be used alone, or two or more of these alkaline proteolytic enzymes may be used in combination.

As the alkaline proteolytic enzyme, a commercially available product can also be used, and examples thereof include: “Alcalase 2.5L” manufactured by Novozyme; “Protin SD-AY10” and “Protease P “Amano” 3SD″ manufactured by Amano Enzyme Inc.; “Multifect PR6L” and “Optimase PR89L” manufactured by Danisco Japan Ltd.; “Sumizyme MP” manufactured by Shin Nihon Chemical Co., Ltd.; “Delvolase” manufactured by DSM Japan K.K.; “Biopurase OP”, “Biopurase SP-20FG”, and “Biopurase SP-4FG” manufactured by Nagase ChemteX Corporation; “Orientase 22BF” manufactured by HBI Enzymes Inc.; and “AROASE XA-10” manufactured by Yakult Pharmaceutical Industry Co., Ltd.

According to an embodiment of the present invention, the optimum pH of the alkaline proteolytic enzyme is not particularly limited, provided that the alkaline proteolytic enzyme has activity in an alkaline environment, but is, for example, 8.0 to 14.0, preferably 8.0 to 12.0, more preferably 8.0 to 10.0, even more preferably 8.0 to 9.0, and most preferably 8.5.

According to an embodiment of the present invention, the optimum temperature of the alkaline proteolytic enzyme is not particularly limited, but is preferably not higher than 60° C., and more preferably not higher than 50° C., from the viewpoint of not requiring undue heating and accordingly preventing a thermal change (pyrolysis) of the PHA. The lower limit of the optimum temperature is not particularly limited, but is preferably not lower than room temperature (e.g., 25° C.), from the viewpoint of not requiring an undue cooling operation and therefore being economical.

According to an embodiment of the present invention, the disruption and solubilization of the cell-derived component in the step (a) can be carried out with use of lysozyme and alcalase in combination.

A duration of the above enzyme treatment in the step (a) can vary according to the type, pH, temperature, and/or the like of the enzyme, but is, for example, one hour to eight hours, and preferably two hours to six hours.

Note that a solvent (the “solvent” may also be referred to as an “aqueous medium”) contained in the aqueous PHA suspension in the present production method may be water or a mixed solvent of water and an organic solvent. In the mixed solvent, the concentration of the organic solvent, which is compatible with water, is not particularly limited, provided that the concentration is equal to or lower than the solubility, in water, of the organic solvent used. The organic solvent which is compatible with water is not particularly limited, and examples thereof include: alcohols such as methanol, ethanol, 1-propanol, 2-propanol, 1-butanol, 2-butanol, iso-butanol, pentanol, hexanol, and heptanol; ketones such as acetone and methyl ethyl ketone; ethers such as tetrahydrofuran and dioxane; nitriles such as acetonitrile and propionitrile; amides such as dimethylformamide and acetamide; dimethyl sulfoxide; pyridine; and piperidine. Among these examples, methanol, ethanol, 1-propanol, 2-propanol, 1-butanol, 2-butanol, iso-butanol, acetone, methyl ethyl ketone, tetrahydrofuran, dioxane, acetonitrile, propionitrile, and the like are preferable because they are easy to remove. Further, methanol, ethanol, 1-propanol, 2-propanol, butanol, acetone, and the like are more preferable, because they are easy to obtain. Furthermore, methanol, ethanol, and acetone are particularly preferable. Note that the aqueous medium contained in the aqueous PHA suspension may contain another solvent, the cell-derived component, a compound which is produced during purification, and/or the like, provided that the essentials of the present invention are not impaired.

The aqueous medium contained in the aqueous PHA suspension in the present production method preferably contains water. The amount of the water contained in the aqueous medium is preferably not less than 5% by weight, more preferably not less than 10% by weight, even more preferably not less than 30% by weight, and particularly preferably not less than 50% by weight.

### (Other Steps)

According to an embodiment of the present invention, the present production method may include the following steps before the step (a).

<Step (a1)>

A step (a1) is a step of culturing the microbial cell which contains the PHA.

In the step (a1), the microbial cell described in the section <Microbial cell (microorganism)>above is, for example, used.

In the step (a1), a method for culturing the microbial cell is not particularly limited, but examples thereof include the method described in paragraphs 0041 to 0048 of the International Publication No. WO 2019/142717.

<Step (a2)>

A step (a2) is a step of inactivating microbial cells obtained in the step (a1). In this step, the microbial cells obtained in the step (a1) are inactivated to obtain an inactivated culture solution.

In the step (a2), a method for inactivating the microbial cells is not particularly limited, but examples thereof include a method in which a culture solution that contains the microbial cells containing, for example, P3HA is heated and stirred at the temperature of the culture solution of 60° C. to 70° C. for 7 hours.

<Step (a3)>

A step (a3) is a step of adjusting the concentration and pH of the inactivated culture solution obtained in the step (a2). The step (a3) is mainly carried out in a case where the viscosity of the inactivated culture solution obtained in the step (a2) is high, and the concentration and pH of the inactivated culture solution are adjusted so that the viscosity of the inactivated culture solution is reduced. The step (a3) facilitates the solubilization in the step (a).

A method for adjusting the concentration and pH of the inactivated culture solution in the step (a3) is not particularly limited, but the adjustment is carried out by any method used in this technical field. For example, by adding hydrogen peroxide or the like to the inactivated culture solution, it is possible to adjust the concentration of the inactivated culture solution. Examples of a method for adjusting the pH include a method in which a basic compound is added to the inactivated culture solution. The basic compound is not particularly limited, but is preferably an alkali metal hydroxide or an alkaline-earth metal hydroxide, and more preferably sodium hydroxide. One of these basic compounds may be used alone, or two or more of these basic compounds may be used in combination.

### (Step (b))

In the step (b) of the present production method, the aqueous PHA suspension is recovered by centrifugation after the step (a). By the step (b), it is possible to remove the cell-derived impurity (cell wall, protein, or the like) contained in the aqueous PHA suspension.

In the step (b), the recovery of the aqueous PHA suspension is carried out by any centrifugation method publicly known in this technical field. The centrifugation method is not particularly limited, but examples thereof include centrifugation with use of a centrifugal settler, a centrifugal dehydrator, or the like.

Examples of the centrifugal settler include centrifugal settlers of a separation plate type (such as disc type, self-cleaning type, nozzle type, screw decanter type, or skimming type), a cylinder type, and a decanter type. According to a sediment component discharging method, each of the centrifugal settlers is categorized into a batch-wise centrifugal settler or a continuous centrifugal settler. Similarly, the centrifugal dehydrator is categorized into a batch-wise centrifugal dehydrator or a continuous centrifugal dehydrator. By using any of these devices, it is possible to separate a sediment containing the PHA from a culture solution component due to a difference in specific gravity.

Since the amount of the impurities which are to remain in an end product depends mainly on the steps (a) and (b), it is preferable to reduce the impurities to the smallest possible amount. As a matter of course, in some uses, the impurities may be mixed in the end product to the extent that the physical properties of the end product are not impaired.

However, in a case where a highly pure PHA is required for, for example, medical use, it is preferable to reduce the impurities to the smallest possible amount. An index of the degree of purification in reducing the impurities can be, for example, the amount of PHA surface adhesion protein contained in the aqueous PHA suspension. The amount of this protein with respect to the weight of the PHA is preferably not more than 2,000 ppm, more preferably not more than 1,900 ppm, even more preferably not more than 1,800 ppm, and most preferably not more than 1,700 ppm. In a case where the amount of the PHA surface adhesion protein in the aqueous PHA suspension falls within the above range, there is an advantage that the rate of leakage ratio is not excessively high. An inferred reason why this effect is brought about is that, in a case where the amount of the PHA surface adhesion protein is small, PHAs easily gather.

(Step (c′))

In the step (c′), the aqueous PHA suspension recovered by centrifugation usually has a pH greater than 7. In the step (c′) of the present production method, the pH of the aqueous PHA suspension obtained in the step (b) is adjusted to 2.5 to 5.5. Carrying out the pH adjustment of the step (c′) reduces the rate of leakage in the filtration of the step (d).

In the step (c′), the pH of the aqueous PHA suspension is 2.5 to 5.5, preferably 2.6 to 5.0, more preferably 2.7 to 4.5, even more preferably 2.8 to 4.0, and particularly preferably 3.0 to 3.8. In a case where the pH of the aqueous PHA suspension falls within the above range, there is an advantage that, in the filtration step, it is possible to improve the filtrate permeation rate without increasing the rate of leakage of the PHA into the filtrate. An inferred reason why this effect is brought about is that PHAs are not excessively small and thus easily agglutinate. The upper limit of the pH is preferably not more than 5.5, from the viewpoint of reducing coloration during heating and melting of the PHA and from the viewpoint of ensuring the stability of the molecular weight of the PHA during heating and/or drying and preventing a decrease in the molecular weight. The lower limit of the pH is preferably not less than 2.5, from the viewpoint of the acid resistance of a container.

In the step (c′), a method for adjusting the pH is not particularly limited, but examples thereof include a method in which acid is added. The acid is not particularly limited, but may be either an organic acid or an inorganic acid, and may or may not be volatile. More specifically, examples of the acid can include sulfuric acid, hydrochloric acid, phosphoric acid, and acetic acid.

In the step (c′), the aqueous PHA suspension is preferably heated. A heating temperature is not particularly limited, but is, for example, preferably 40° C. to 90° C., more preferably 50° C. to 80° C., and more preferably 60° C. to 70° C. Heating the aqueous PHA suspension in the step (c′) facilitates adjustment of the pH. Therefore, it is possible to obtain a highly thermally stable PHA.

According to an embodiment of the present invention, it is preferable that an additional pH adjustment should not be carried out after the pH adjustment in the step (c) and until after the step (d) is carried out.

### (Step (c))

In the step (c) of the present production method, the aqueous PHA suspension is heat-treated so that the aqueous PHA suspension has a temperature of 60° C. to 120° C. With the step (c), it is possible to increase the filtrate permeation rate during the filtration.

In the step (c), the aqueous PHA suspension is heat-treated so that the aqueous PHA suspension has a temperature of preferably 60° C. to 120° C., more preferably 62° C. to 118° C., and even more preferably 65° C. to 115° C. In a case where the temperature of the aqueous PHA suspension falls within the above range, it is possible to further increase the filtrate permeation rate during the filtration.

A method for heat-treating the aqueous PHA suspension in the step (c) is not particularly limited, but examples thereof include (i) a method in which steam is used to warm the container holding the aqueous PHA suspension, (ii) a method in which oil is used to warm the container holding the aqueous PHA suspension, and (iii) a method in which steam is directly put into the aqueous PHA suspension. The temperatures of the steam in (i) and (iii) and the temperature of the oil in (ii) are not particularly limited, provided that these temperatures cause the aqueous PHA suspension to have a temperature of 60° C. to 120° C. in the step (c). For example, the temperatures are 95° C. to 150° C.

### (Step (e))

In the step (e) of the present production method, the PHA obtained in the step (d) is dried at 20° C. to 100° C. With the step (e), it is possible to evaporate the water contained in the aqueous PHA suspension, and possible to adjust the water content.

A method for drying the PHA in the step (e) is not particularly limited, but examples thereof include: heating; vacuum drying; and drying at normal temperature. The drying is preferably carried out by heating, from the viewpoint of a moderate drying speed. A heating medium (e.g., hot air or a jacket) during the drying is preferably at 30° C. to 90° C., more preferably at 40° C. to 80° C., and even more preferably at 50° C. to 70° C.

### (Step (f))

In the step (f) of the present production method, the dried PHA is redispersed in an aqueous solvent to obtain an aqueous PHA suspension. By carrying out the step (f) after the step (e), obtained is an aqueous PHA suspension containing the PHA which has a particle diameter substantially the same as the original particle diameter (primary particle diameter).

In the step (f), a method for redispersing the PHA is not particularly limited, and the redispersion is carried out by any method used in this technical field.

In the step (f), the volume median diameter of the PHA is not particularly limited, provided that the volume median diameter is substantially the same as the volume median diameter of the PHA in the step (a). For example, the volume median diameter is preferably 0.5 μm to 5.0 μm, more preferably 1.0 μm to 4.5 μm, and even more preferably 1.0 μm to 3.0 μm. As used herein, the expression “volume median diameter is substantially the same” means that the difference from the volume median diameter of the PHA in the step (a) is not more than 1.0 μm.

The steps (e) and (f) may be carried out successively. That is, the PHA dried in the step (e) may be redispersed in the step (f) to obtain the aqueous PHA suspension. According to an embodiment of the present invention, the present production method includes: a step of drying, at 20° C. to 100° C., the polyhydroxyalkanoate obtained in the filtration step; and a step of redispersing the dried polyhydroxyalkanoate in an aqueous solvent to obtain an aqueous polyhydroxyalkanoate suspension which contains the polyhydroxyalkanoate having a volume median diameter of 0.5 μm to 5.0 μm.

### [3. PHA Agglutinate]

The water content of the present PHA agglutinate is 5.0% to 25.0% (W. B.). Note that the PHA agglutinate may also be referred to as a “PHA cake”, a “filter cake” or a “PHA filter cake”.

The water content of the present PHA agglutinate is 5.0% to 25.0% (W. B.), preferably 5.5% to 23.0% (W. B.), more preferably 6.0% to 21.0% (W. B.), even more preferably 6.5% to 20.0% (W. B.), and particularly preferably 7.0% to 19.0% (W. B.). In a case where the water content of the present PHA agglutinate falls within the above range, the PHA agglutinate becomes not a slurry state but a solid state, and thus there is an advantage that the PHA agglutinate is easy to put into a dryer. Note that the water content of the present PHA agglutinate is measured by the method described in Examples.

According to an embodiment of the present invention, the present PHA agglutinate is produced by the present production method.

The present PHA agglutinate may contain various components which have been produced in the course of the present production method or could not have been removed, provided that the present PHA agglutinate has the effect of the present invention.

The present PHA agglutinate can be used for various purposes such as paper, films, sheets, tubes, plates, rods, containers (e.g. bottle containers), bags, and components.

The present invention is not limited to the embodiments, but can be altered by a skilled person in the art within the scope of the claims. The present invention also encompasses, in its technical scope, any embodiment derived by combining technical means disclosed in differing embodiments.

That is, aspects of the present invention include the following.

<1>A method for producing a PHA, including:
a filtration step of filtering an aqueous PHA suspension having a pH of 2.5 to 5.5, by filter-pressing with use of a filter medium having an air permeability of 0.1 cm³/cm²/min to 2.5 cm³/cm²/min,
the aqueous PHA suspension having a liquid density of 0.50 g/mL to 1.08 g/mL in the filtration step,
the filtration step including a compression step and an air blow step.

<2> The method as described in <1>, wherein a filtrate has a solid content concentration of not more than 1,000 mg/L in the filtration step.

<3> The method as described in <1> or <2>, wherein the filter medium is not precoated.

<4> The method as described in any one of <1> through <3>, wherein the aqueous PHA suspension has a temperature of 20° C. to 95° C. in the filtration step.

<5> The method as described in any one of <1> through <4>, further including a step (a) of disrupting and solubilizing a cell-derived component of a microbial cell which contains a PHA, the cell-derived component being a component other than the PHA,
the PHA having a volume median diameter of 0.5 μm to 5.0 μm in the step (a).

<6> The method as described in <5>, further including, after the step (a), a step (b) of recovering the aqueous PHA suspension by centrifugation.

<7> The method as described in any one of <1> through <6>, wherein a polyhydroxyalkanoate obtained in the filtration step is dried at 20° C. to 100° C.

<8> The method as described in any one of <1> through <7>, wherein an air blow pressure in the air blow step is 0.01 MPa to 1.5 MPa.

<9> The method as described in any one of <1> through <8>, wherein an air blow duration in the air blow step is 1 minute to 50 minutes.

<10>A method for producing an aqueous polyhydroxyalkanoate suspension, including a step of dispersing, in an aqueous solvent, a polyhydroxyalkanoate produced by a method described in any one of <1> through <9>.

<11>A PHA agglutinate having a water content of 5.0% to 25.0% (W. B.).

### EXAMPLES

The following description will discuss the present invention in more detail on the basis of Examples. However, the present invention is not limited to Examples. In Examples, “P3HB3HH” is used as the “PHA”. The term “PHA” in Examples can therefore be read as “P3HB3HH”.

### [Measurement Method]

Measurements in Examples and Comparative Examples were carried out by the following methods.

### (Solid Content Concentration of Filtrate)

An appropriate amount of a filtrate was vacuumed with use of a glass filter and with use of a glass filter paper GS-25 (manufactured by ADVANTEC). Subsequently, a solid content concentration was calculated from the mass of the filter paper which had been dried.

### (Water Content of PHA Agglutinate)

A dehydrated cake produced in a test was cut into three or nine pieces so as to collect an appropriate amount of a piece from the dehydrated cake. Subsequently, the piece was dried in a constant-temperature drier at 105° C. for approximately 15 hours, and the water content of a PHA agglutinate was calculated from the difference in mass of the piece between before and after drying.

### (Air Permeability)

An air permeability was measured by the method described in JIS L 1096. Specifically, with use of a Frazier type permeameter (Permeameter P2, manufactured by Toyo Seiki Seisaku-sho, Ltd.), the amount of air suctioned was adjusted so that an inclined manometer indicates 125 Pa, and then an air flow rate was measured in such a situation.

### (Liquid Density)

An aqueous PHA suspension immediately before the filtration step was heated to a filtration temperature, and then 20 mL of the aqueous PHA suspension was suctioned into a 20-mL plastic syringe (manufactured by Terumo Corporation) the weight of which had been measured in advance. Next, the weight of the 20 mL of the aqueous suspension and the plastic syringe was measured, and a liquid density at the filtration temperature was calculated by dividing, by the volume (20 mL) of the liquid, a weight (g) obtained by subtracting the weight of the 20-mL plastic syringe from the measured weight.

### (Volume Median Diameter)

The volume median diameter of a PHA was measured with use of a laser diffraction/scatter particle size distribution meter LA-950 manufactured by HORIBA.

### (Filtration Temperature)

As the temperature during filtration, a temperature immediately before the aqueous PHA suspension was put into a filter was measured with use of a type-K thermocouple (AD5601A, manufactured by A&D Company, Limited).

(pH of aqueous PHA suspension in step (c′))

A pH was measured with use of a pH meter (9652-10D, manufactured by HORIBA). The pH was measured at a position in the aqueous PHA suspension, the position being the farthest from a position where acid was added, the aqueous PHA suspension having been brought into a fluidized state with use of a stirring blade or the like. For example, in a case where acid was added at the wall surface of a container, the pH was measured at the center of the container.

### EXAMPLE 1

### (Preparation of Microbial Cell Culture Solution)

Ralstonia eutropha described in the International Publication No. WO 2019/142717 was cultured by the method described in paragraphs 0041 to 0048 of the same document, so that a microbial cell culture solution containing microbial cells which contained a PHA was obtained. Note that Ralstonia eutropha is currently classified as Cupriavidus necator. The composition ratio of repeating units (the composition ratio of a 3HB unit to a 3HH unit) of the PHA (P3HB3HH) in the microbial cell culture solution was 94/6 (mol/mol).

### (Inactivation)

The microbial cell culture solution obtained as described above was heated and stirred at the temperature of the microbial cell culture solution of 60° C. to 70° C. for 7 hours for sterilization. An inactivated culture solution was thus obtained.

### (Viscosity Reduction Treatment)

To the inactivated culture solution obtained as described above, 35% by weight hydrogen peroxide (manufactured by FUJIFILM Wako Pure Chemical Corporation) was added such that the hydrogen peroxide was 1% by weight of the inactivated culture solution. Next, a 30% aqueous sodium hydroxide solution was added so that the pH of the inactivated culture solution was adjusted to 11.0. By keeping on adding the 30% aqueous sodium hydroxide solution while maintaining the solution at 60° C., the pH of the solution was maintained at 11.0 for 180 minutes. An aqueous PHA suspension was thus obtained.

### (Enzyme Treatment)

To the aqueous PHA suspension obtained as described above, 95% sulfuric acid was added so that the pH of the aqueous PHA suspension was adjusted to 7.0±0.2. The solid content concentration of the aqueous PHA suspension having sulfuric acid added thereto was measured, and found to be 30% by weight. After the addition of the sulfuric acid, Lysozyme (manufactured by FUJIFILM Wako Pure Chemical Corporation), which is an enzyme that degrades sugar chains (peptidoglycan) in a cell wall, was added so that the concentration thereof in the aqueous PHA suspension was 10 ppm, and the aqueous PHA suspension was kept at 50° C. for 2 hours. After that, Alcalase 2.5L (manufactured by Novozyme), which is a proteolytic enzyme, was added so that the concentration thereof in the aqueous PHA suspension was 300 ppm. Then, 30% sodium hydroxide was added at 50° C. so that the pH of the aqueous PHA suspension was adjusted to 8.5. While such adjustment was carried out, the pH was maintained for 2 hours.

### (Alkaline Treatment)

Sodium dodecyl sulfate (SDS, manufactured by Kao Corporation) was added to the above enzyme-treated solution so as to be 0.3% by weight of the enzyme-treated solution. After that, the pH of the enzyme-treated solution was adjusted to 11.0±0.2 with use of an aqueous sodium hydroxide solution. Next, the enzyme-treated solution was centrifuged (4,000 G, 10 minutes), and a supernatant was then removed. A twofold concentrated aqueous PHA suspension was thus obtained. Subsequently, the following operation was repeated 4 times: adding, to this concentrated aqueous PHA suspension, sodium hydroxide in an amount equal to that of the removed supernatant; centrifuging the aqueous PHA suspension again (4,000 G, 10 minutes); and removing a supernatant. The volume median diameter of the PHA was 2.2 μm. The amount of protein in the aqueous PHA suspension thus obtained was 1,000 ppm.

### (pH Adjustment)

The solid content concentration of the aqueous PHA suspension obtained as described above was adjusted to 25% by weight, and the aqueous PHA suspension was then kept at 60° C. Next, the pH was adjusted to 3.5 by adding 10% sulfuric acid. The liquid density was 1.00 g/mL.

### (Filtration)

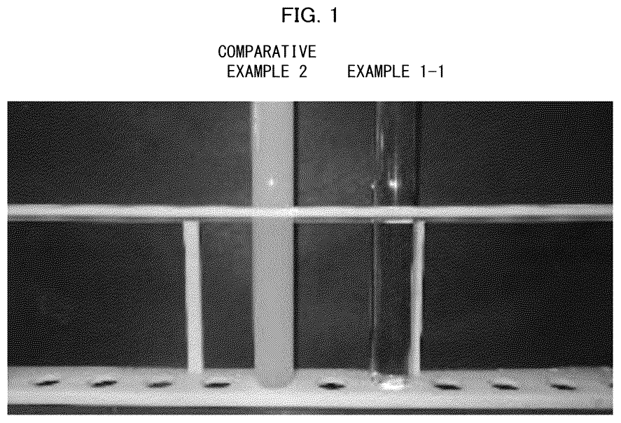

The aqueous PHA suspension was put in a water bath at 63° C., heated so as to have a temperature of 60° C., and filtered with use of a filter press (ISD type 360, manufactured by ISHIGAKI COMPANY, LTD.). A filter cloth having an air permeability of 1.0 cm³/cm²/min (IP196C-1, manufactured by ISHIGAKI COMPANY, LTD.) was used. Note that the filter cloth used was not precoated. The filtration was carried out at a compression pressure of 0.7 MPa, and air blow was carried out at an air blow pressure adjusted to 0.2 MPa to 0.6 MPa. Table 1 shows the solid content concentration of an obtained filtrate and the water content of a PHA agglutinate. FIG. 1 shows a photograph of the liquid having passed through the filter cloth in Example 1-1.

### COMPARATIVE EXAMPLE 1

A PHA agglutinate was obtained in the same manner as that in Example 1, except that the air blow was not carried out in the filter-pressing filtration. Table 1 shows the solid content concentration of an obtained filtrate and the water content of the PHA agglutinate.

TABLE 1

| | | | Solid content | Water content |
|---|---|---|---|---|
| | Air blow | Air blow | concentration | of PHA |
| | pressure | duration | of filtrate | agglutinate |
| | [MPa] | [min] | [mg/L] | [% (W. B.)] |
| Example 1-1 | 0.2 | 24 | Not more than 10 | 17.4 |
| Example 1-2 | 0.3 | 25 | Not more than 10 | 12.2 |
| Example 1-3 | 0.4 | 15 | Not more than 10 | 11.5 |
| Example 1-4 | 0.6 | 13 | Not more than 10 | 9.7 |
| Comparative | None | — | Not more than 10 | 33.1 |
| Example 1 | | | | |

### EXAMPLE 2

An enzyme-treated solution was obtained in a manner similar to that in Example 1, except for the operations following the enzyme treatment. SDS was added to the enzyme-treated solution so as to be 0.2% by weight of the enzyme-treated solution. After that, the pH of the enzyme-treated solution was adjusted to 11.0±0.2 with use of an aqueous sodium hydroxide solution. Next, the enzyme-treated solution was centrifuged (4,000 G, 10 minutes), and a supernatant was then removed. A twofold concentrated aqueous PHA suspension was thus obtained. Subsequently, the following operation was repeated twice: adding, to this concentrated aqueous PHA suspension, sodium hydroxide in an amount equal to that of the removed supernatant; centrifuging the aqueous PHA suspension again (4,000 G, 10 minutes); and removing a supernatant. The amount of protein in the aqueous PHA suspension thus obtained was 3,000 ppm. The volume median diameter of a PHA was 2.2 μm. The pH adjustment step and the filtration step were each carried out under a condition similar to that in Example 1-2, and a filter cake was obtained. The liquid density after the pH adjustment step was 1.00 g/mL. The water content (W. B.) of the obtained cake was 13.1%, and the solid content concentration of a liquid having passed through a filter cloth was not more than 10 mg/L.

### EXAMPLE 3

### (Preparation of Microbial Cell Culture Solution)

Ralstonia eutropha described in the International Publication No. WO 2019/142717 was cultured by the method described in paragraphs 0041 to 0048 of the same document, so that a microbial cell culture solution containing microbial cells which contained a PHA was obtained. The composition ratio of repeating units (the composition ratio of a 3HB unit to a 3HH unit) of the PHA (P3HB3HH) in the microbial cell culture solution was 80/20 to 88/12 (mol/mol), and more accurately 85/15 (mol/mol). Filtration was carried out with use of the obtained microbial cell culture solution in a manner similar to that in Example 1, except that lysozyme was not added in the enzyme treatment step. Note that the volume median diameter of the PHA after the alkaline treatment step was 2.3 μm. The amount of protein in an aqueous PHA suspension thus obtained was 1,800 ppm. The liquid density after the pH adjustment step was 1.00 g/mL.

TABLE 2

| | | | Solid content | Water content |
|---|---|---|---|---|
| | Air blow | Air blow | concentration | of PHA |
| | pressure | duration | of filtrate | agglutinate |
| | [MPa] | [min] | [mg/L] | [% (W. B.)] |
| Example 3-1 | 0.3 | 15 | Not more than 10 | 13.0 |
| Example 3-2 | 0.4 | 15 | Not more than 10 | 12.3 |

### COMPARATIVE EXAMPLE 2

Filter-pressing filtration was carried out in the same manner as that in Comparative Example 1, except for a filter cloth having an air permeability of 3.0 cm³/cm²/min (IP126C, manufactured by ISHIGAKI COMPANY, LTD.) was used. FIG. 1 shows a photograph of a liquid having passed through the filter cloth. It is found that the filter cloth having an air permeability of 1.0 cm³/cm²/min did not allow the particles to pass therethrough, but the filter cloth having an air permeability of 3.0 cm³/cm²/min allowed the particles to pass therethrough. The solid content concentration of a filtrate was 2,060 mg/L.

### EXAMPLE 4

The PHA agglutinate obtained in Example 1-3 was placed in a dryer (WFO-700, manufactured by EYELA) and dried at 60° C. for 24 hours. The dried PHA agglutinate was redispersed in water, and the solid content concentration was adjusted to 15% by weight. The pH was adjusted between 7 and 9 with use of a 1% NaOH aqueous solution and a 1% H₂SO₄ aqueous solution, and the PHA agglutinate redispersed in the water was then stirred. An aqueous PHA suspension was thus prepared. The particle diameter of PHA particles in the aqueous PHA suspension was measured after 30 minutes of stirring, and the volume median diameter was found to be 2.8 μm.

### SUMMARY

It was found from the above that the present production method makes it possible to considerably reduce the water content of a PHA agglutinate.

### INDUSTRIAL APPLICABILITY

With the present production method, it is possible to produce a PHA having a low water content through a simple operation. Thus, it is possible to advantageously use the present production method in production of a PHA.

Furthermore, it is possible to suitably use the present PHA agglutinate in the fields of agriculture, fishery, forestry, horticulture, medicine, sanitary products, clothing, non-clothing, packaging, automobiles, building materials, and the like.

1. A method for producing a polyhydroxyalkanoate, the method comprising:
filtering an aqueous polyhydroxyalkanoate suspension having a pH of from 2.5 to 5.5 by filter-pressing using a filter medium having an air permeability of from 0.1 cm³/cm²/min to 2.5 cm³/cm²/min,
wherein the aqueous polyhydroxyalkanoate suspension has a liquid density of from 0.50 g/mL to 1.08 g/mL in the filtering, and
filtrating comprises compressing and air blowing.

2. The method of claim 1, wherein a filtrate has a solid content concentration of 1,000 mg/L or less in the filtrating.

3. The method of claim 1, wherein the filter medium is not precoated.

4. The method of claim 1, wherein the aqueous polyhydroxyalkanoate suspension has a temperature of from 20° C. to 95° C. in the filtrating.

5. The method of claim 1, further comprising:
(a) disrupting and solubilizing a cell-derived component of a microbial cell which comprises a polyhydroxyalkanoate, wherein the cell-derived component is a component other than the polyhydroxyalkanoate,
wherein the polyhydroxyalkanoate has a volume median diameter of from 0.5 μm to 5.0 μm in the (a).

6. The method of claim 5, further comprising, after the (a), (b) recovering the aqueous polyhydroxyalkanoate suspension by centrifugation.

7. The method of claim 1, further comprising drying a polyhydroxyalkanoate obtained in the filtering at a temperature of from 20° C. to 100° C.

8. The method of claim 1, wherein an air blow pressure in the air blow is from 0.01 MPa to 1.5 MPa.

9. The method of claim 1, wherein an air blow duration in the air blow is from 1 minute to 50 minutes.

10. A method for producing an aqueous polyhydroxyalkanoate suspension, the method comprising:
redispersing, in an aqueous solvent, a polyhydroxyalkanoate produced by the method of claim 1.

11. (canceled)
